(19) United States
(12) Patent Application Publication
(10) Pub. No.: US 2007/0181708 A1
(43) Pub. Date: Aug. 9, 2007

(54) Die cut air freshener

(76) Inventor: Rhonda C. Garrett, Greenbrier, AR (US)

Correspondence Address: JAMES RAY & ASSOCIATES, 2640 PITCAIRN ROAD, MONROEVILLE, PA, 15146, US

(21) Appl. No.: 11/348,674

(22) Filed: Feb. 7, 2006

(51) Int. Cl. A61L 9/04; A24F 25/00

(52) U.S. Cl. 239/56; 239/53; 239/34; 239/54

(57) ABSTRACT

A method of making a hanging type air freshener for use in at least one of a vehicle and a preselected room of at least one of a residential home and an office. The method includes the steps of selecting a shape for such hanging type air freshener to be formed into and further selecting a scent to be used for such hanging type air freshener. A carrier material is selected for the scent selected and such scent is then impregnated into such carrier material selected. The carrier material is then die cut into the shape selected and the hanging type air freshener is then packaged for shipment.

### FIELD OF THE INVENTION

The present invention relates, in general, to an air freshener, and, more specifically the present invention relates to hanging type air freshener which is die cut and which can be used in an automobile or in a home.

### BACKGROUND OF THE INVENTION

There are a variety of air fresheners on the market today and they vary from liquids in a container to solid deodorizers.

U.S. Pat. No. 5,164,178 of Muysson provides a solid granular deodorant that consists of porous granular calcium sulfate base (CaSO_{4.}2H₂O), a portion of essential oils absorbed on the porous granular base and micro capsules filled with another portion of essential oils.

Brizard in U.S. Pat. No. 5,492,675 provides a deodorant system which includes a compact container for an odor absorbent, which may be temporarily secured to another surface such as an interior wall of a refrigerator or a garbage container, and which includes a calendar to assist in determining a replacement date for the deodorant system.

Vogt et al. In U.S. Pat. No. 6,767,521 provides a system for dehumidifying and deodorizing, the system comprises a hanging, dehumidifying and deodorizing pouch comprising a housing having an impermeable membrane and a semi-permeable securely attached to the impermeable membrane. The housing enclosing moisture absorbent material and deodorizing material.

### SUMMARY OF THE INVENTION

Thus, the present invention provides in a first aspect a method of making a hanging type air freshener for use in at least one of a vehicle and a preselected room of at least one of a residential home and an office. The method includes the steps of selecting a shape for such hanging type air freshener to be formed into and further selecting a scent to be used for such hanging type air freshener. A carrier material is selected for the scent selected and such scent is then impregnated into such carrier material selected. The carrier material is then die cut into the shape selected and the hanging type air freshener is then packaged for shipment.

In a second aspect, the present invention provides a hanging type air freshener for use in at least one of a vehicle and a preselected room of at least one of a residential home and an office. Such hanging type air freshener comprising a carrier material die cut into a predetermined shape. A preselected scent is impregnated into such predetermined shape which is then placed into a package for each of preserving said predetermined scent and shipping said hanging type air freshener.

### OBJECTS OF THE INVENTION

It is, therefore, one of the primary objects of the present invention to provide a method of making a hanging type air freshener which can be formed into many different shapes and have several different scents.

Another object of the present invention is to provide a hanging type air freshener which is inexpensive to manufacture.

These and various other objects and advantages of this invention will become apparent after a full reading of the following detailed description, particularly, when read in conjunction with the attached drawings as described below and the appended claims.

### BRIEF DESCRIPTION OF THE DRAWINGS

FIG. 1 is a partial perspective view of the present invention in the shape of a cell phone.

FIG. 2 is a partial perspective view of the present invention in the shape of an automobile.

FIG. 3 is a partial perspective view of the present invention in the shape of a football headgear.

FIG. 4 is a partial perspective view of the present invention in the shape of an animal.

### BRIEF DESCRIPTION OF THE PRESENTLY PREFERRED AND ALTERNATE EMBODIMENTS OF THE INVENTION

Prior to proceeding with the more detailed description of the present invention it should be noted that, for the sake of clarity, identical components which have identical functions have been designated by identical reference numerals throughout the several views illustrated in the drawings.

The present invention is designed to help eliminate malodorous vehicle interiors or home interiors. The innovative air freshener is intended as a decorative ands useful way to restore a clean smelling vehicle while having such deodorizing material contained in a container with a variety of shapes from team mascots, fancy cars, sports equipment or any other similar type articles.

Thus, the present invention provides in a first aspect a method of making a hanging type air freshener for use in at least one of a vehicle and a preselected room of at least one of a residential home and an office. The method includes the steps of selecting a shape for such hanging type air freshener to be formed into and further selecting a scent to be used for such hanging type air freshener. A carrier material is selected for the scent selected and such scent is then impregnated into such carrier material selected. The carrier material is then die cut into the shape selected and the hanging type air freshener is then packaged for shipment.

The shape that is selected in a previous step is selected from the group consisting of animals, school logos, business logos, vehicles, boats, trains, sporting equipment, buildings, phones, famous sites and structures. The animals can be selected from pets and wild animals. Such wild animals would be such animals normally found in a zoo.

The pets cold be selected from dogs, cats, birds, fish, and even animals from the rodent family such as a gerbil.

The scent used in this method is generally selected from the group consisting of spices, fruit extracts, trees and new car smells, while the carrier for such scent is selected from paper products. It is preferred if such paper products are recycled paper products.

Such scent can be impregnated by means of dipping, spraying and brushing, while it is preferred that such impregnating be done by dipping.

The method includes the additional step of applying advertising material to at least one side of the hanging air freshener. Such advertising can very well be applied to both sides of the air freshener.

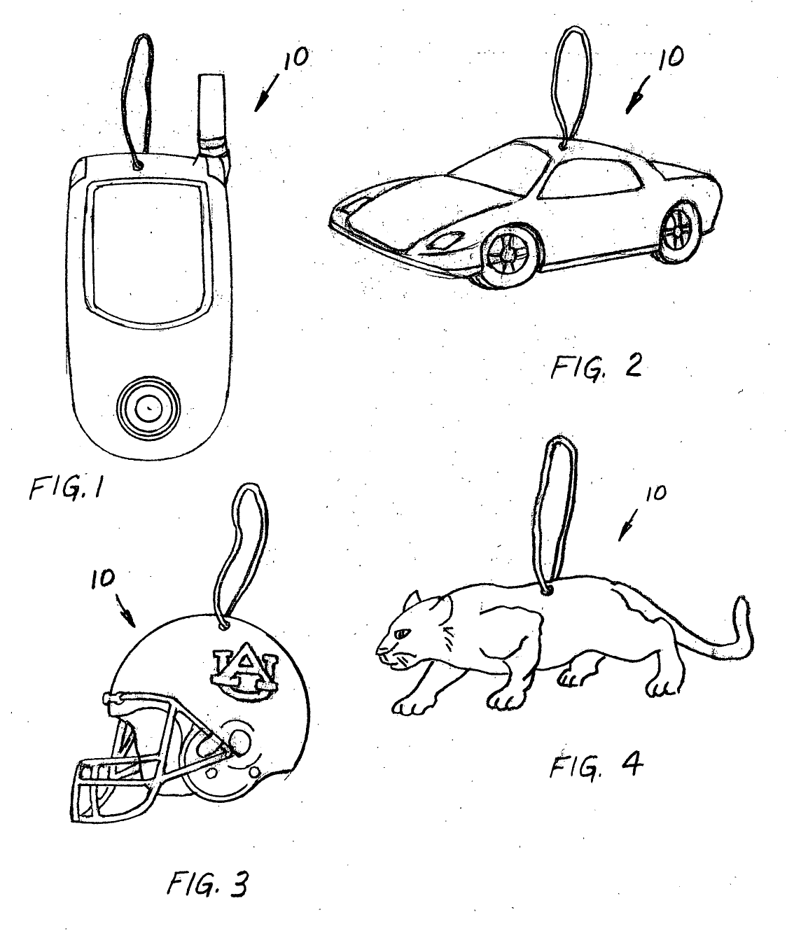

FIGS. 1-4 show some of the various shapes that could be used in the present invention. These Figures only show a few of the many possibilities that could be used to form the air freshener. As is evident in FIGS. 3 and 4 such air freshener could be designed to incorporate a team logo or team mascot or nickname. It could be used for high school teams, college or professional teams and could be color matched to fit the colors of the school or team.

In a second aspect, the present invention provides a hanging type air freshener, generally designated 10, for use in at least one of a vehicle and a preselected room of at least one of a residential home and an office. Such hanging type air freshener 10 comprising a carrier material die cut into a predetermined shape. A preselected scent is impregnated into such predetermined shape which is then placed into a package for each of preserving said predetermined scent.

As described previously such predetermined shape of said hanging type air freshener is selected from the group consisting of animals, school logos, business logos, vehicles, boats, trains, sporting equipment, buildings, phones, famous sites and structures.

The scent for such air freshener is selected from the group consisting of spices, fruit extracts, trees and new car smells, while it is preferred that the carrier material for such scents be selected from paper products and even more preferred that such paper products are recycled paper products

Such hanging type air freshener further includes advertising material on at least one side of the hanging type air freshener and could include advertising material on both sides of the hanging type air freshener.

The air freshener wherein the predetermined shape is determined or correlated to an end use of the hanging type air freshener, that is, if it is designed for a college the shape could be a panther as the school's nickname whereas if it is designed as a promo for a car dealer the air freshener could be in the shape of a car.

While a presently preferred embodiment and alternate embodiments of the present invention has been described in detail above, it should be understood that various other adaptations and/or modifications of the invention can be made by those persons who are particularly skilled in the art without departing from either the spirit of the invention or the scope of the appended claims.

I claim:

1. A method of making a hanging type air freshener for use in at least one of a vehicle and a preselected room of at least one of a residential home and an office, said method comprising the steps of:
(a) selecting a shape said hanging type air freshener is to be formed into;
(b) selecting a scent to be used for said hanging type air freshener;
(c) selecting a carrier material for said scent selected in step (b);
(d) impregnating said scent selected in step (b) into said carrier selected in step (c);
(e) die cutting said carrier into said shape selected in step (a); and
(f) packaging for shipment said hanging type air freshener cut in step (e).

2. A method, according to claim 1, wherein said shape selected in step (a) is selected from the group consisting of animals, school logos, business logos, vehicles, boats, trains, sporting equipment, buildings, phones, famous sites and structures.

3. A method, according to claim 2, wherein said animals are selected from pets and wild animals.

4. A method, according to claim 3, wherein said wild animals are animals normally found in a zoo.

5. A method, according to claim 3, wherein said pets are selected from dogs, cats, birds, fish, and animals from the rodent family.

6. A method, according to claim 1, wherein said scent is selected from the group consisting of spices, fruit extracts, trees and new car smells.

7. A method, according to claim 1, wherein said carrier material is selected from paper products.

8. A method, according to claim 7, wherein said paper product selected is a recycled paper product.

9. A method, according to claim 1, wherein step (d) includes dipping, spraying and brushing.

10. A method, according to claim 9, wherein step (d) is accomplished by dipping.

11. A method, according to claim 1, wherein said method includes the additional step of applying advertising material to at least one side of said hanging air freshener.

12. A method, according to claim 11, wherein said method includes the additional step of applying advertising material to each side of said hanging air freshener.

13. A hanging type air freshener for use in at least one of a vehicle and a preselected room of at least one of a residential home and an office, said hanging type air freshener comprising:
(a) a carrier material die cut into a predetermined shape;
(b) a preselected scent impregnated into said predetermined shape; and
(c) a package for each of preserving said predetermined scent and shipping said hanging type air freshener.

14. A hanging type air freshener, according to claim 13, wherein said predetermined shape of said hanging type air freshener is selected from the group consisting of animals, school logos, business logos, vehicles, boats, trains, sporting equipment, buildings, phones, famous sites and structures.

15. A hanging type air freshener, according to claim 13, wherein said preselected scent of said hanging type air freshener is selected from the group consisting of spices, fruit extracts, trees and new car smells.

16. A hanging type air freshener, according to claim 13, wherein said carrier material is selected from paper products.

17. A hanging type air freshener, according to claim 16, wherein said paper product selected is a recycled paper product.

18. A hanging type air freshener, according to claim 13, wherein said hanging type air freshener further includes advertising material on at least one side of said hanging type air freshener.

19. A hanging type air freshener, according to claim 18, wherein said hanging type air freshener includes advertising material on each side of said hanging type air freshener.

20. A hanging type air freshener, according to claim 18, wherein said predetermined shape of said hanging type air freshener is correlated to an end use of said hanging type air freshener.
